(19) United States
(12) Patent Application Publication
(10) Pub. No.: US 2002/0052402 A1
(43) Pub. Date: May 2, 2002

(54) Composition and method

(75) Inventors: Steven Curtis Zicker, Lawrence, KS (US); Karen J. Wedekind, Meriden, KS (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 09/922,633

(22) Filed: Aug. 6, 2001

Related U.S. Application Data
(60) Provisional application No. 60/244,511, filed on Oct. 31, 2000.
(60) Provisional application No. 60/253,447, filed on Nov. 28, 2000.

(51) Int. Cl. A61K 31/385; A61K 31/34; A61K 31/355

(52) U.S. Cl. 514/440; 514/474; 514/458

(57) ABSTRACT

A composition comprising an antioxidant or mixture thereof in quantities sufficient to

prevent, or

inhibit, or

reverse

deterioration of cognitive function.

### BACKGROUND OF THE INVENTION

As humans age their mental faculties appear to lessen. This applies to humans in general and is not related to specific conditions such as Alzheimer's and other known-specific disease conditions affecting mental capacity. Memory is not as good as it once was. Learning certain tasks can take a longer time. These types of mental changes are some times associated with or referred to as “old age”. Fairly recently this general area is known as cognitive function deterioration and in more generic terms as diminished mental capacity.

This deterioration of cognitive function in older humans can now be prevented, inhibited or treated with specific components. These components can be administered in the diet or taken separately orally.

### SUMMARY OF THE INVENTION

In accordance with the invention there is a composition comprising an antioxidant or mixture thereof quantities sufficient to

a. prevent or

b. inhibit, or

c. reverse

deterioration of cognitive function,

A further aspect is a method of preventing, inhibiting or reversing deterioration of cognitive function through the administration of this composition.

### DETAILED DESCRIPTION OF THE INVENTION

Humans can start having deterioration in cognitive function as early as about 50 but generally it can become noticeable at 60, or 70 years of age.

As used in this application, the component(s) that accomplish this are an antioxidant or mixture thereof. An antioxidant is a material that quenches a free radical. Examples of such materials are beta-carotene, selenium, coenzyme Q10 (ubiquinone), lutein, tocotrienols, soy isoflavones, S-adenosylmethionine, glutathione, taurine, N-acetylcysteine, Vitamin E, Vitamin C, alpha-lipoic acid, l-carnitine and the like. Vitamin E can be administered as a tocopherol or a mixture of tocopherols and various derivatives thereof such as esters like Vitamin E acetate, succinate, palmitate, and the like. The alpha form is preferable but beta, gamma and delta forms can be included. The d form is preferable but racemic mixtures are acceptable. The forms and derivatives will function in a Vitamin E like activity after ingestion. Vitamin C can be administered as ascorbic acid and its various derivatives thereof such as calcium phosphate salts, cholesteryl salt, 2-monophosphate, and the like which will function in a vitamin C like activity after ingesting by the human. They can be in any form such as liquid, semisolid, solid and heat stable form. Alpha-lipoic acid can be administered as alpha lipoic acid or as a lipoate derivative as in U.S. Pat. No. 5,621,117, racemic mixtures, salts, esters or amides thereof. L-carnitine and various derivatives of carnitine such as the salts such as the hydrochloride, fumarate and succinates, as well as acetylated carnitine, and the like can be used.

The quantities administered to the human are calculated as the active material, per se, that is measured as specific material. The minimum amounts employed should not bring about toxicity. Vitamin E minimum is about 50 or 100 or 150 mg/day. Generally, no more than about 2,000 or desirably about 1,000 or about 750 mg/day can be employed. Vitamin C minimum quantities are generally about 150, about 200 or about 250 mg/day. Maximums generally do not exceed about 1,000 mg/day but can be lower, for example, about 900 or about 750 mg/day. Alpha lipoic acid can be administered up to about 10 or 20 mg/kg body weight/day. Minimums are about 0.5 or 1 mg/kg body weight/day. L-carnitine is administered in the same manner as alpha lipoic acid. Alpha lipoic acid and l-carnitine can be administered at much higher levels but there is no substantially different efficacy observed.

Additional antioxidants and their quantities are given in mg/kg body weight/day numbers.

Beta-carotene at least about 0.02 mg/kg body weight/day 1-15 ppm can be employed.

Selenium at about 0.02 mg/kg body weight/day up to 5 ppm can be employed.

Lutein at least about 5 ppm mg/kg body weight/day can be employed.

Tocotrienols at least about 0.5 mg/kg body weight/day can be employed.

Coenzyme Q10 at least about 0.5 mg/kg body weight/day can be employed.

S-adenosylmethionine at least about 1.0 mg/kg body weight/day can be employed.

Taurine at least about 10 mg/kg body weight/day can be employed.

Soy isoflavones at least about 0.5 mg/kg body weight/day can be used.

N-acetylcysteine at least about 1.0 mg/kg/body weight/day can be used.

Glutathione at least about 1.0 g/kg body weight/day can be used.

Ginkgo Biloba at least 1.0 mg/kg body weight/day can be used.

The quantities can be administered in a typical “one a day” dosage similar to a vitamin. It can be administered in tablet, capsule, caplet for example, or in a liquid carrier form all for ingestion by the human. Additionally, the daily dosage can be broken down into two, three, or four dosage unit forms for convenience or other reasons. Additionally, it can be administered in a food such as snack bars, cereals, and the like.

### EXAMPLE 1

A supplement or food providing 750 mg vitamin E, 250 mg/vitamin C, 200 mg lipoic acid and 400 mg of l-carnitine is eaten by an adult human on a daily basis for 5 years starting at age 45. By the age of 50 no signs of mental deterioration such as disorientation, memory loss, learning impairment, or ability to cope independently with their environmental living situation, classified by objective (or nonobjective) tests such as Folstein's Mini-mental State Exam (MME), Logical Memory and Visual Reproduction tests from the Wechsler Memory Scale (WMS), Katz' scale of activities of daily living, Lawton's scale of instrumental activities of daily living, Pfeiffer's mental status questionnaire, or other pertinent tests as diagnosed by themselves, a care-giver, or health care provider, are observed.

### EXAMPLE 2

An adult human having signs of mental deterioration, such as disorientation, memory loss, learning impairment, or ability to cope independently with their environmental living situation, as classified by objective (or nonobjective) tests such as Folstein's Mini-mental State Exam (MME), Logical Memory and Visual Reproduction tests from the Wechsler Memory Scale (WMS), Katz' scale of activities of daily living, Lawton's scale of instrumental activities of daily living, Pfeiffer's mental status questionnaire, or other pertinent tests as diagnosed by themselves, a care-giver, or a health care provider, is provided a daily food or supplement providing 750 mg vitamin E, 250 mg/vitamin C, 200 mg lipoic acid and 400 mg of l-carnitine on a daily basis for 1 year. After one year of dosing the above signs are significantly reduced.

### EXAMPLE 3

An adult human having signs of mental deterioration such as disorientation, memory loss, learning impairment, or ability to cope independently with their environmental living situation, as classified by objective (or nonobjective) tests such as Folstein's Mini-mental State Exam (MME), Logical Memory and Visual Reproduction tests from the Wechsler Memory Scale (WMS), Katz' scale of activities of daily living, Lawton's scale of instrumental activities of daily living, Pfeiffer's mental status questionnaire, or other pertinent tests as diagnosed by themselves, a care-giver, or a health care provider, is provided a daily food or supplement providing 750 mg vitamin E, 250 mg/vitamin C, 200 mg lipoic acid and 400 mg of l-carnitine on a daily basis for 2 years. After 2 years of dosing the aforementioned signs have essentially disappeared.

What is claimed is:

1. A composition comprising an antioxidant or mixture thereof in quantities sufficient to
a. prevent, or
b. inhibit, or
c. reverse
deterioration of cognitive function.

2. The composition in accordance with claim 1 wherein deterioration of cognitive function is prevented.

3. The composition in accordance with claim 1 wherein deterioration of cognitive function is inhibited.

4. The composition in accordance with claim 1 wherein deterioration of cognitive function is reversed.

5. The composition in accordance with claim 1 wherein Vitamin E is at least about 50 mg/day, Vitamin C is at least about 150 mg/day, l-carnitine is at least about 0.5 mg/kg body weight/day, and alpha lipoic acid is at least about 0.5 mg/kg body weight/day.

6. A method for preventing or inhibiting or reversing deterioration of cognitive function in a human which comprises administering a composition of claim 1 to said human.

7. The method in accordance with claim 6 wherein the human is in need of said administering.

8. The method in accordance with claim 6 wherein the deterioration of cognitive function is prevented.

9. The method in accordance with claim 6 wherein the deterioration of cognitive function is inhibited.

10. The method in accordance with claim 6 wherein the deterioration of cognitive function is reversed.
